(19) United States
(12) Patent Application Publication
(10) Pub. No.: US 2026/0053755 A1
(43) Pub. Date: Feb. 26, 2026

(54) PHARMACEUTICAL COMPOSITIONS FOR USE IN TREATING OBSTRUCTIVE SLEEP APNEA

(71) Applicant: Caliway Biopharmaceuticals Co., Ltd., New Taipei City (TW)

(72) Inventor: Yu-Fang LING, New Taipei City (TW)

(21) Appl. No.: 19/307,526

(22) Filed: Aug. 22, 2025

Related U.S. Application Data
(60) Provisional application No. 63/685,849, filed on Aug. 22, 2024.

(51) Int. Cl. A61K 31/121; A61K 31/05; A61P 11/00; A61P 25/00

(52) U.S. Cl. CPC A61K 31/121; A61K 31/05; A61P 11/00; A61P 25/00

(57) ABSTRACT

A method for alleviating a symptom associated with obstructive sleep apnea (OSA), the method comprising administering to a subject in need thereof an effective amount of a pharmaceutical composition, which comprises: (a) a resveratrol compound and/or a curcumin compound; and (b) a pharmaceutically acceptable carrier.

### CROSS REFERENCE TO RELATED APPLICATIONS

This application claims the benefit of the filing date of U.S. Provisional Application No. 63/685,849, filed Aug. 22, 2024, the entire contents of which is incorporated by reference herein.

### BACKGROUND

Sleep apnea is a type of sleep disorders characterized by repeated stop and start of breathing during sleep. Obstructive sleep apnea (OSA) is the most common form of sleep apnea. It occurs when throat muscles relax and block the flow of air into the lungs. Symptoms of obstructive sleep apnea include loud or frequent snoring or silent pauses in breathing and choking or gasping sounds. Patients with obesity are more likely to be affected by sleep apnea. Uncontrolled OSA is directly tied to an increased risk in cardiovascular and metabolic health.

Current treatment of OSA includes continuous positive airway pressure (CPAP) therapy, oral appliance therapy (OAT) or surgery. Both CPAP and OAT require a patient to wear a device during sleep, such as a face mask connected by tubing to a constantly running machine or a mouth guard-like device to maintain an open and unobstructed airway. These treatment options are expensive with low patient compliance. Surgical options include a variety of procedures, all involving sides effects and varied rates of success.

It is therefore of great interest to develop effective, low cost, and convenient therapeutic options for alleviating OSA.

### SUMMARY

The present disclosure provides a method for alleviating a symptom associated with obstructive sleep apnea (OSA), the method comprising administering to a subject in need thereof an effective amount of a pharmaceutical composition, which comprises: (a) a resveratrol compound (e.g., resveratrol) and/or a curcumin compound (e.g., curcumin); and (b) a pharmaceutically acceptable carrier. In some embodiments, the pharmaceutical composition comprises the resveratrol compound (e.g., resveratrol). In other embodiments, the pharmaceutical composition comprises the curcumin compound (e.g., curcumin). In yet other embodiments, the pharmaceutical composition comprises both the resveratrol compound (e.g., resveratrol) and the curcumin compound (e.g., curcumin).

In some embodiments, the pharmaceutically acceptable carrier in any of the compositions disclosed herein may comprise a first pharmaceutically acceptable non-ionic surfactant having a hydrophilic-lipophilic balance value (HLB value) greater than 9. Examples include, but are not limited to, Polyoxyl 40 Stearate, Polyoxyl 20 Cetostearyl Ether, Polyoxyl 12 Cetostearyl Ether, Tween 80, polyoxyl 15 hydroxystearate (solutol HS 15), a polyoxyethylene derivative, a polyoxyethylene castor oil derivative, or a combination thereof.

In some embodiments, the first pharmaceutically acceptable non-ionic surfactant forms a first plurality of micelles with the resveratrol compound and/or the curcumin compound. In some examples, the weight ratio of the resveratrol compound and/or the curcumin compound to the first pharmaceutically acceptable non-ionic surfactant in the composition is 1:2-1:500. In some examples, the micelles in the first plurality of micelles have a diameter ranging from about 1 nm to about 250 nm. In some instances, the micelles in the first plurality of micelles have a diameter ranging from 5 nm to about 50 nm. Alternatively, or in addition, the micelles in the first plurality of micelles may have a polydispersity index (PDI) value less than 0.4. In some specific examples, the first plurality of micelles encompasses both the resveratrol compound (e.g., resveratrol) and the curcumin compound (e.g., curcumin).

In some embodiments, the pharmaceutically acceptable carrier further comprises a second pharmaceutically acceptable non-ionic surfactant having a hydrophilic-lipophilic balance value (HLB value) greater than 9. Examples include, but are not limited to, Polyoxyl 40 Stearate, Polyoxyl 20 Cetostearyl Ether, Polyoxyl 12 Cetostearyl Ether, Tween 80, polyoxyl 15 hydroxystearate (solutol HS 15), a polyoxyethylene derivative, a polyoxyethylene castor oil derivative, or a combination thereof. The second pharmaceutically acceptable non-ionic surfactant may be identical to the first pharmaceutically acceptable non-ionic surfactant. Alternatively, the second pharmaceutically acceptable non-ionic surfactant may be different from the first pharmaceutically acceptable non-ionic surfactant.

In some instances, the micelles in the second plurality of micelles have a diameter ranging from about 1 nm to about 250 nm. In some instances, the micelles in the second plurality of micelles have a diameter ranging from 5 nm to about 50 nm. Alternatively, or in addition, the micelles in the second plurality of micelles may have a polydispersity index (PDI) value less than 0.4.

In some examples, the first plurality of micelles is formed by the first pharmaceutically acceptable non-ionic surfactant and the resveratrol compound (e.g., resveratrol) and the second pharmaceutically acceptable non-ionic surfactant and the curcumin compound (e.g., curcumin) form a second plurality of micelles. In some instances, the weight ratio of the curcumin compound to the second pharmaceutically acceptable non-ionic surfactant in the composition is 1:2-1:500.

In some embodiments, the pharmaceutical composition further comprises a cosolvent, a suspending agent, an oil phase excipient, or a combination thereof.

In some embodiments, the subject for the treatment is a human patient suffering from OSA. Exemplary symptoms associated with OSA comprises loud and disruptive snoring, witnessed apneas during sleep, excessive daytime sleeping, morning headaches, waking during the night and gasping or choking, awakening in the morning with a dry mouth and/or sore throat. In some examples, the pharmaceutical composition is administered to the subject via injection (e.g., subcutaneous injection). In some instances, the pharmaceutical composition is administrated to the tongue, e.g., via injection such as subcutaneous injection. In other instances, the pharmaceutical composition is administrated to a perioral aera, e.g., by sublingual administration.

Also provided herein is any of the pharmaceutical compositions disclosed herein for use in alleviating symptoms of OSA (e.g., treating OSA), as well as use of such a pharmaceutical composition for manufacturing a medicament for use in alleviating OSA in a subject.

The details of one or more embodiments of the invention are set forth in the description below. Other features or advantages of the present invention will be apparent from the following drawings and detailed description of several embodiments, and also from the appended claims.

### DETAILED DESCRIPTION

Obstructive sleep apnea (OSA) is a prevalent disease characterized by cyclical obstruction of the upper airway, which would result in intermittent cessation or reduction of airflow during sleep. Kim et al., J. Clinical Medicine, 2019, 8:2049. Multiple factors are reported for contributing to the development of, including impaired upper airway anatomy, impaired upper airway neuromuscular responses, respiratory control instability, and low respiratory arousal threshold. Current treatments for OSA either require expensive devices during sleep, which result in low patient compliance, or involve invasive surgical procedurals.

The present disclosure provides compositions and methods for treating and/or alleviating symptoms of obstructive sleep apnea.

### I. Pharmaceutical Compositions

The pharmaceutical compositions for use in the instant disclosure comprise a resveratrol compound (e.g., resveratrol), a curcumin compound (e.g., curcumin), or a combination thereof, and a pharmaceutically acceptable carrier, which may comprise one or more pharmaceutically acceptable non-ionic surfactants such as those disclosed herein.

### A. Resveratrol Compounds

In some embodiments, the pharmaceutical compositions provided herein comprise one or more resveratrol compounds. As used herein, a resveratrol compound refers to a compound having a core structure of:

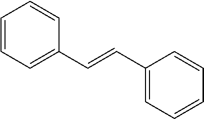 [SMILES: C(=C\c1ccccc1)/c1ccccc1]

with suitable substitutions at one or more positions, for example, on either or both of the ring structures. Suitable substitutions include halogen, thiol, hydroxyl, C₁₋₄ alkyl, C₂₋₄ alkenyl, OR₅, COOR₆, or —OC(═O)R₇, wherein each of R₅, R₆, and R₇ are independently H, or C₁₋₄ alkyl (e.g., methyl). Exemplary C₁₋₄ alkyl groups include, but are not limited to, methyl, ethyl, n-propyl, isopropyl, n-butyl, sec-butyl, isobutyl, tert-butyl. Exemplary C₂₋₄alkenyl groups include, but are not limited to, ethenyl, propenyl, butenyl; alkynyl; aralkyl; halogenated alkyl; heteroalkyl; aryl; heterocyclyl; cycloalkyl; cycloalkenyl; cycloalkynyl.

As used herein, the term “alkyl” refers to a linear, saturated, acyclic, monovalent hydrocarbon radical or branched, saturated, acyclic, monovalent hydrocarbon radical, having from one to three carbon atoms attached to the rest of the molecule by a single bond, e.g., methyl, ethyl, n-propyl, or 1-methylethyl (iso-propyl). An optionally substituted alkyl radical is an alkyl radical that is optionally substituted, valence permitting, by one, two, three, four, or five substituents independently selected from the group consisting of halo, cyano, nitro, oxo, hydroxyl, thio, or amino.

As used herein, the term “alkenyl” refers to a linear, acyclic, monovalent hydrocarbon radical or branched, acyclic, monovalent hydrocarbon radical, containing a carbon-carbon double bond, having two or three carbon atoms attached to the rest of the molecule by a single bond, e.g., ethenyl, or propenyl. An optionally substituted alkenyl radical is an alkenyl radical that is optionally substituted, valence permitting, by one, two, or three substituents independently selected from the group consisting of: halo, cyano, nitro, hydroxyl, thio, or amino. As described herein, an alkene may be a Z or E alkene. An alkene shown as a Z alkene include the E isomer of the alkene.

“Amino” refers to a radical of the formula —NRbRc where Rb and Rc are each hydrogen, or an alkyl radical as defined above containing one to three carbon atoms. The alkyl part of the optionally substituted amino radical is optionally substituted as defined above for an alkyl radical.

“Thiol” refers to a radical of the formula —SRd where Rd is a hydrogen or an alkyl radical as defined above containing one to three carbon atoms. The alkyl part of the optionally substituted thiol radical is optionally substituted as defined above for an alkyl radical.

In some examples, the resveratrol compound may be resveratrol, having the structure of

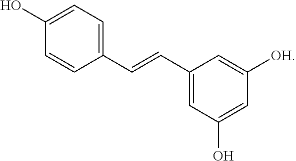 [SMILES: Oc1ccc(/C=C/c2cc(O)cc(O)c2)cc1]

In other examples, the resveratrol compound may be a resveratrol derivative, for example, hydroxylated resveratrol, methoxylated resveratrol, glycosylated resveratrol, or oligomers of resveratrol.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Tnterscience, New York, 1981); Wilen et al., Tetrahedron 33:2725 (1977); Eliel, Stereochemistiy of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

### B. Curcumin Compounds

In some embodiments, the pharmaceutical compositions provided herein comprise one or more curcumin compounds. As used herein, a curcumin compound refers to a compound having a core structure of:

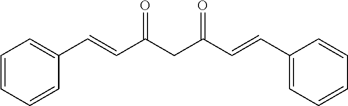 [SMILES: O=C(/C=C/c1ccccc1)CC(=O)/C=C/c1ccccc1]

with suitable substitutions at one or more positions, for example, on either or both of the ring structures. Suitable substitutions include halogen, thiol, hydroxyl, C₁₋₄ alkyl, C₂₋₄ alkenyl, OR₅, COOR₆, or —OC(═O)R₇, wherein each of R₅, R₆, and R₇ are independently H, or C₁₋₄ alkyl (e.g., methyl). Exemplary C₁₋₄ alkyl groups include, but are not limited to, methyl, ethyl, n-propyl, isopropyl, n-butyl, sec-butyl, isobutyl, tert-butyl. Exemplary C₂₋₄ alkenyl groups include, but are not limited to, ethenyl, propenyl, butenyl; alkynyl; aralkyl; halogenated alkyl; heteroalkyl; aryl; heterocyclyl; cycloalkyl; cycloalkenyl; cycloalkynyl.

In some examples, the curcumin compound may be curcumin, having the structure of

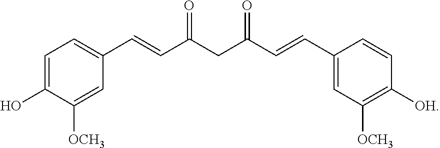 [SMILES: COc1cc(/C=C/C(=O)CC(=O)/C=C/c2ccc(O)c(OC)c2)ccc1O]

In other examples, the curcumin compound may be a curcumin derivative, for example, hydroxylated curcumin, methoxylated curcumin, glycosylated curcumin, or oligomers of curcumin. In some instances, curcumin derivatives can be produced by chemical reaction between aryl-aldehydes and acetylacetone.

### C. Pharmaceutical Compositions

Any of the resveratrol compounds, any of the curcumin compounds, or a combination thereof as disclosed here may be mixed with one or more pharmaceutically acceptable carriers to form a pharmaceutical composition, which can be used for alleviating one or more symptoms of OSA in a subject who needs the treatment. In some embodiments, the pharmaceutical composition provided herein comprises one or more resveratrol compounds such as resveratrol. In other embodiments, the pharmaceutical composition comprises one or more curcumin compounds. In yet other embodiments, the pharmaceutical composition comprises both a resveratrol compound such as resveratrol and a curcumin compound such as curcumin.

Pharmaceutically acceptable carriers include diluents, fillers, salts, buffers, stabilizers, solubilizers and other material which are well-known in the art. Exemplary pharmaceutically acceptable carriers in particular are described in U.S. Pat. No. 5,211,657. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from a suitable inorganic base, (e.g., sodium hydroxide, barium hydroxide, iron (ii) hydroxide, iron (III) hydroxide, magnesium hydroxide, calcium hydroxide, aluminium hydroxide, ammonium hydroxide, potassium hydroxide, caesium hydroxide, or lithium hydroxide) or a suitable organic base (e.g., pyridine, methyl amine, imidazole, benzimidazole, histidine, phosphazene bases, or a hydroxide of an organic cation such as quaternary ammonium hydroxide and phosphonium hydroxide). Also, pharmaceutically-acceptable salts can be prepared as alkaline metal or alkaline earth salts, such as lithium, sodium, potassium or calcium salts.

The pharmaceutical compositions as described herein can comprise pharmaceutically acceptable carriers, excipients, or stabilizers in the form of lyophilized formulations or aqueous solutions. Remington: The Science and Practice of Pharmacy 20th Ed. (2000) Lippincott Williams and Wilkins, Ed. K. E. Hoover. Such carriers, excipients or stabilizers may enhance one or more properties of the active ingredients in the compositions described herein, e.g., bioactivity, stability, bioavailability, and other pharmacokinetics and/or bioactivities.

Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; benzoates, sorbate and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, serine, alanine or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ (polysorbate), PLURONICS™ (nonionic surfactants), or polyethylene glycol (PEG).

In some embodiments, the pharmaceutically acceptable carriers may comprise one or more non-ionic surfactant having a hydrophilic-lipophilic balance value (HLB value) greater than 9. In some examples, the pharmaceutically acceptable carriers may further comprise, a solvent, a buffer agent, an oil phase (e.g., medium-chain triglycerides, or soybean oil), or a combination thereof.

In some embodiments, the pharmaceutically acceptable carriers may comprise the one or more non-ionic surfactants, which may form nanoparticles with the resveratrol compound (e.g., resveratrol), the curcumin compound (e.g., curcumin), or both. Alternatively, the pharmaceutical composition provided herein may contain no nanoparticles.

In some embodiments, the pharmaceutically acceptable carriers may further comprise a co-surfactant, a co-solvent, an antioxidant, or any combination thereof. In some instances, the pharmaceutically acceptable carriers may comprise a lipophilic solvent, e.g., triacetin, medium-chain triglycerides, or oil. In some examples, the pharmaceutical composition provided herein may be free of water. Alternatively, or in addition, the pharmaceutical composition provided herein may be free of antioxidants.

### Non-Ionic Surfactants

The pharmaceutical composition disclosed herein may comprise one or more non-ionic surfactants, which may form nanoparticles with the active agent and/or the hydrophilic therapeutic agent as disclosed herein. Non-ionic surfactants used in the present technology are beneficial at least in the formation of nanoparticles in the composition. In some embodiments, nanoparticles have the effect of encapsulating the active agent and/or the hydrophilic therapeutic agent.

Non-ionic surfactants used in any of the pharmaceutical compositions disclosed herein preferably have a hydrophilic-lipophilic balance (HLB) value greater than 5. The non-ionic surfactant is used in the present technology in a ratio as set forth above. Exemplary non-ionic surfactants include, but are not limited to, polysorbate 80 (Tween® 80), polyoxyl 15 hydroxystearate (Solutol® HS-15), polyoxyethylene castor oil derivatives (e.g., polyoxyl 35 castor oil (Kolliphor® ELP), polyoxyl 40 hydrogenated castor oil (Koliphor® RH40) and polyoxyl 60 hydrogenated castor oil (Cremophor® RH60), Polyoxyethylene (12) glyceryl laurate (UNIGLY ML-212), Polyoxyl 20 Stearate (Myrj™ S20), Polyoxyl 40 Stearate (Myrj™ S40), Polyoxyl 12 Cetostearyl Ether (Kolliphor® CS 12) and Polyoxyl 20 Cetostearyl Ether (Kolliphor® CS 20).

In some instances, the non-ionic surfactant may be a polyoxyethylene derivative, also known as Pegylated excipient. In some examples, the polyoxyethylene derivative is a PEG castor oil derivative, which are materials obtained by reacting varying amounts of ethylene oxide with either castor oil or hydrogenated castor oil. Examples include PEG-35 castor oil and PEG-40 hydrogenerated Castor Oil. In other examples, the polyoxyethylene derivative is a PEG ester, which can be manufactured by reacting a polyethylene glycol with a fatty acid. Examples include PEG-40 Stearate and PEG-15 Hydroxystearate. In some instances, the ester may be a sorbitan fatty acid ester (e.g., PEG-20 sorbitan monooleate, PEG-40 sorbitan monooleate, PEG-60 sorbitan monooleate, PEG-80 sorbitan monooleate, PEG-20 sorbitan isostearate, PEG-30 sorbitan tetraoleate, PEG-40, -60 sorbitan tetraoleate, PEG-40 sorbitan diisostearate, or PEG-60 sorbitan tetrastearate), an alkyl glyceride (e.g., PEG-8 Caprylic/capric glycerides, PEG-32 hydrogenated palm glycerides, or PEG-32 Lauroyl glycerides), or an alkyl ether (e.g., PEG-6 cetostearyl ether, PEG-12 cetostearyl ether, PEG-20 cetostearyl ether, PEG-10 cetyl ether, PEG-20 cetyl ether, PEG-4 lauryl ether, PEG-23 lauryl ether, PEG-2 oleyl ether, PEG-10 oleyl ether, PEG-20 oleyl ether, PEG-2 stearyl ether, PEG-10 stearyl ether, PEG-21 stearyl ether, or PEG-100 stearyl ether). In yet other instances, the ester may be a laurate (e.g., PEG-2 laurate, PEG-4 laurate, PEG-6 laurate, PEG-8 laurate. PEG-9-14 laurate, PEG-20 laurate, PEG-32-150 laurate, or PEG-12 glyceryl laurate), a dilaurate (e.g., PEG-2 dilaurate, PEG-4 dilaurate, or PEG-6-150 dilaurate), or a stearate (e.g., PEG-2 stearate, PEG-3 stearate, PEG-4 stearate, PEG-4 isostearate, PEG-5-7 stearate, PEG-6-8 isostearate, PEG-8 stearate, PEG-9 stearate, PEG-10 stearate, PEG-10-isostearate, PEG-12 isostearate, PEG-12-18 stearate, PEG-20 stearate, PEG-23-45 stearate, PEG-40 stearate, PEG-50 stearate, PEG-100 stearate, PEG-75-150 stearate, or PEG-6 and PEG-32 palmitostearate), a glyceryl stearate (e.g., Glyceryl stearate/PEG-40 stearate, Glyceryl stearate/PEG-100 stearate, PEG-120 glyceryl stearate, PEG-20 methyl glucose sesquistearate, or PEG-25 propylene glycol stearate), a distearate (e.g., PEG-2 distearate, PEG-3-120 distearate, PEG-150 distearate, or PEG-175 distearate), a hydroxystarate (e.g., PEG-15 hydroxystearates).

In some instances, the non-ionic surfactant may be a castor oil derivative (e.g., PEG-35 castor oil or PEG-40 castor oil) or a hydrogenated castor oil (e.g., PEG-40 hydrogenated castor oil, PEG-54 hydrogenated castor oil, or PEG-60 hydrogenated castor oil).

Additional examples of non-ionic surfactants include PEG-15 cocamine, Vitamin E polyethylene glycol succinate, PEG-75 lanolin, and PEG-120 methyl glucose dioleate.

### Nanoparticles and Preparations Thereof

In some embodiments, the pharmaceutical compositions disclosed herein may comprise nanoparticles formed by one or more non-ionic surfactants as disclosed herein and the resveratrol compound such as resveratrol, the curcumin compound such as curcumin, or both.

In some instances, the pharmaceutical composition provided herein may comprise nanoparticles such as micelles formed by a non-ionic surfactant as those provided herein and a resveratrol compound such as resveratrol. In other instances, the pharmaceutical composition provided herein may comprise nanoparticles such as micelles formed by a non-ionic surfactant as those provided herein and a curcumin compound such as curcumin. In yet other instances, the pharmaceutical composition provided herein may comprise nanoparticles such as micelles formed by a non-ionic surfactant as those provided herein, a resveratrol compound such as resveratrol, and a curcumin compound such as curcumin. For example, the pharmaceutical composition may comprise one population of nanoparticles such as micelles comprising the non-ionic surfactant and both of the resveratrol compound and the curcumin compound. In other examples, the pharmaceutical composition may comprise two populations of nanoparticles such as micelles, one comprising a first non-ionic surfactant and the resveratrol compound and the other population comprising a second non-ionic surfactant and the curcumin compound. The first and second non-ionic surfactants may be identical. Alternatively, the first and second non-ionic surfactant may be different.

In some embodiments, the nanoparticles may comprise the non-ionic surfactant and the resveratrol and/or curcumin compound at a suitable weight ratio, which lead to suitable particle sizes and/or suitable polydispersity index (PDI) values. For example, a suitable weight ratio between the resveratrol and/or curcumin compound and the non-ionic surfactant may range from about 1:1-1:1000, for example, between about 1:1-1:800, between about 1:1-1:600, between about 1:1-1:500, between about 1:1-1:200, or between about 1:1-1:100. In other examples, the suitable weight ratio between the resveratrol and/or curcumin compound and the non-ionic surfactant ranges from 1:5 to 1:1000, for example, between 1:5-1:150, between 1:5-1:200, between 1:5-1:300, between 1:5-1:400, between 1:5-1:500, between about 1:8-1:10, between about 1:8-1:20, between about 1:8-1:40, between about 1:8-1:100, between 1:8-1:150, between 1:8-1:200, between 1:8-1:300, between 1:8-1:400, between 1:8-1:500, between about 1:10-1:20, between about 1:10-1:40, between about 1:10-1:100, between 1:10-1:150, between 1:10-1:200, between 1:10-1:300, between 1:10-1:400, between 1:10-1:500, between about 1:20-1:40, between about 1:20-1:100, between 1:20-1:150, between 1:20-1:200, between 1:20-1:300, between 1:20-1:400, between 1:20-1:500, between about 1:40-1:100, between 1:40-1:150, between 1:40-1:200, between 1:40-1:300, between 1:40-1:400, between 1:40-1:500, between 1:100-1:150, between 1:100-1:200, between 1:100-1:300, between 1:100-1:400, between 1:100-1:500, or between 1:150-1:500.

In some instances, the pharmaceutical compositions disclosed herein may comprise nanoparticles having an average size of up to 200 nm, for example, up to 150 nm, up to 100 nm, up to 50 nm, or up to 20 nm. In some examples, the average size of the nanoparticles may range from 5-200 nm, for example, 5-150 nm, 5-100 nm, 5-50 nm, or 5-20 nm. In some examples, the average size of the nanoparticles may range from 1-200 nm, for example, 1-150 nm, 1-100 nm, 1-50 nm, or 1-20 nm.

Alternatively, or in addition, the nanoparticles in the pharmaceutical compositions may have a suitable PDI value, for example, up to 0.4. In some examples, the PDI value may range from 0.05-0.4, e.g., 0.05-0.35, 0.05-0.3, 0.05-0.25, 0.05-0.2, 0.05-0.15, or 0.05-0.1.

Any of the nanoparticles disclosed herein can be prepared by conventional methods or as disclosed herein. One example is provided below. A suitable amount of an active agent or a hydrophilic therapeutic agent may be mixed with a suitable solvent (e.g., alcohols (e.g., methanol, ethanol, propanol), acetonitrile, chlorinated solvents (e.g., dichloromethane, chloroform) diethyl ether, and ethyl acetate) and the mixture can be stirred (e.g., at 150-500 rpm) at a suitable temperature until the active agent or the hydrophilic therapeutic agent as disclosed herein is full dissolved in the solvent to form a solution. A suitable amount of a pharmaceutically acceptable non-ionic surfactant (e.g., those disclosed herein) can be added to the solution. The resultant mixture can be stirred (e.g., at 100-300 rpm) under a suitable temperature to volatilize the solvent. Once the solvent is completely volatilized, a suitable amount of a pharmaceutically acceptable aqueous solution (e.g., normal saline) can be added to the mixture to produce micelles having the active agent or hydrophilic agent encapsulated. The resultant particles can be filtered through a suitable filter (e.g., a 0.2 μm filter) and the filtered solution comprising drug-containing micelles can be stored in the dark in a refrigerator for future use.

Additional details on the preparation of the micelles of the present technology can be found in U.S. Pat. No. 10,610,496, the relevant disclosures of which are incorporated by reference herein for the subject matter and purpose referenced herein.

### Additional Components of Pharmaceutical Compositions

In embodiments, the pharmaceutical composition disclosed herein may further comprise a co-solvent (e.g., to increase the solubility of drugs), a suspending agent (e.g., to reduce the sedimentation rate of drugs), an oil phase excipient (e.g., to increase the stability of the pharmaceutical composition and the solubility of drugs), an antimicrobial preservative, or a combination thereof.

In some instances, the co-solvent can comprise polyethylene glycol, propylene glycol, ethanol, and other co-solvents, or a combination thereof. Alternatively, or in addition, the suspending agent may comprise sodium alginate, glycerol, carboxymethyl cellulose sodium, mannitol, and other suspending agents, or a combination thereof.

In some instances, the oil phase excipient may comprise unsaturated fatty acids, glycerol, triglycerides, and other oil phase excipients, or a combination thereof. For example, the oil phase excipient may comprise one or more unsaturated fatty acids, which may be oleic acid, castor oil, sesame oil, cottonseed oil, soybean oil, safflower oil, corn oil, and other unsaturated fatty acids, or a combination thereof. In some examples, the oil phase excipient may comprise triglycerides, which may be medium chain triglycerides.

In some embodiments, the pharmaceutical composition disclosed herein may comprise a local anesthetic. Examples include, but are not limited to, amides, para-aminobenzoic acid esters, and amino ethers, or a combination thereof. In some examples, the local anesthetic comprises amides, which may bedibucaine, lidocaine, mepivacaine HCl, bupivacine HCl, pyrrocaine HCl, Prilocaine HCl, digammacaine, and oxethazaine, or a combination thereof. In other examples, the local anesthetic comprises para-aminobenzoic acid esters, which may be butacaine, dimethocaine, and tutocaine, or a combination thereof. In yet other examples, the local anesthetic comprises amino ethers, which may be quinisocaine and pramocaine, or a combination thereof.

Alternatively, or in addition, the pharmaceutical composition disclosed herein may comprise one or more antioxidant. Examples include, but are not limited to, beta-carotene, lutein, lycopene, bilirubin, vitamin A, vitamin C (ascorbic acid), vitamin E, citric acid, sodium thiosulfate, Propyl gallate, uric acid, nitric oxide, nitroxide, pyruvate, catalase, superoxide dismutase, glutathione peroxidases, N-acetyl cysteine, and naringenin, or a combination thereof. Alternatively, the pharmaceutical composition is free of antioxidants.

Alternatively, or in addition, the pharmaceutical compositions provided herein may further comprise a second active agent, which is not a resveratrol and/or curcumin compound disclosed herein. In some instances, the second active agent may be encapsulated in nanoparticles.

In some embodiments, the second active agent may comprise a hydrophilic therapeutic agent, for example, green tea extract, epicatechin, epicatechin gallate, epigallocatechin, gallocatechin gallate, gallocatechin, catechin gallate, catechin, epigallocatechin gallate (EGCG), caffeine, carnitine, L-carnitine, synephrine, chlorogenic acid, or a combination thereof.

In some embodiments, the pharmaceutical compositions described herein may be formulated as a topical formulation, for example, a cream, lotion, or gel for topical application. Such cream, lotion, or gel may be formulated using ingredients known in the art to be appropriate for topical medications. In some examples, the topical formation may be in the form of a transdermal patch.

In other embodiments, the pharmaceutical compositions described herein may be formulated as an injectable formulation (e.g., for intradermal injection). A sterile injectable composition, e.g., a sterile injectable aqueous or oleaginous suspension, can be formulated according to techniques known in the art using suitable dispersing or wetting agents (such as Polysorbate 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

In some embodiments, the pharmaceutical composition is in a form of non-oral dosage form, and the subject is a human. For example, the composition formulated for injection can be in the form of powder (e.g., lyophilized powder), sterilized suspension, injectable solution, injectable emulsion, or intravenous fluid. In some examples, the composition maybe placed in a microneedle device for injection. Alternatively, the composition may be formulated for transdermal administration, e.g., in a form of ointment, lotion, liniment, cream, gel, dressing, emulsion, film, patch, poultice, cataplasm, or topical powder.

### II. Methods for Alleviating Obstructive Sleep Apnea

Any of the pharmaceutical compositions disclosed herein, comprising the resveratrol compound such as resveratrol, the curcumin compound such as curcumin, or both, can be used to alleviate one or more symptoms associated with OSA, for example, to treat, delay the onset, or alleviate OSA.

OSA is a sleep-related breathing disorder, which occurs when the throat muscles relax and block the airway during sleep. Typical symptoms associated with OSA include loud snoring, observed episodes of stopped breathing during sleep, waking during the night and gasping or choking, awakening in the morning with a dry mouth or sore throat, morning headaches, or a combination thereof. OSA patients may also suffer from excessive daytime sleepiness, trouble focusing during the day, mood changes, such as depression or being easily upset, and/or high blood pressure.

OSA may be diagnosed via a routine medical procedure. For example, OSA can be diagnosed using a polysomnography (PSG) or sleep study to monitor a patient's breathing, heart rate, brain waves, eye and leg movement, and blood oxygen levels during sleep.

To practice the method disclosed herein, an effective amount of the pharmaceutical composition described herein can be administered to a subject (e.g., a human) in need of the treatment via a suitable route, for example, orally, sublingually, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

In some embodiments, an effective amount of the pharmaceutical composition may be administered to a subject such as a human subject having OSA or exhibiting at least one symptom associated with OSA by a suitable route, for example, injection (e.g., subcutaneous injection), or sublingual administration.

As used herein, “an effective amount” refers to the amount of each active agent required to confer therapeutic effect on the subject, either alone or in combination with one or more other active agents. Determination of whether an amount of the pharmaceutical composition achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of a target disease/disorder. Alternatively, sustained continuous release formulations may be appropriate. Various formulations and devices for achieving sustained release are known in the art.

For the purpose of the present disclosure, the appropriate dosage of the pharmaceutical composition as described herein will depend on the specific active agents and optionally hydrophilic agents employed, the type and severity of the disease/disorder, whether the composition is administered for preventive or therapeutic purposes, previous therapy, the patient's clinical history and response to the active agents, and the discretion of the attending physician. Typically, the clinician will administer a pharmaceutical composition disclosed herein, until a dosage is reached that achieves the desired result.

In some embodiments, the desired result is the reduction or elimination of one or more symptoms associated with OSA (e.g., those disclosed herein). Methods of determining whether a dosage resulted in the desired result would be evident to one of skill in the art. The particular dosage regimen such as dose, timing and repetition, used in the method described herein will depend on the particular subject and that subject's medical history. Treatment efficacy for the target disease/disorder can be assessed by methods well-known in the art.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that delays or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Any of the pharmaceutical compositions disclosed herein may be administered to the subject at least 1 time for a suitable period of time (e.g., once, twice, three times, or four times), for example, 1 week to 1 year. Duration of the treatment may depend on the treatment results.

### III. Kits for Use in Alleviating Obstructive Sleep Apnea

The present disclosure also provides kits for use in treating or alleviating symptoms of obstructive sleep apnea (OSA) in a subject (e.g., a human patient) who needs the treatment. Such kits can include one or more containers comprising the pharmaceutical composition disclosed herein. In some instances, the pharmaceutical composition may be co-used with a second therapeutic agent.

In some embodiments, the kit can comprise instructions for use in accordance with any of the methods described herein. The included instructions can comprise a description of administration of the pharmaceutical composition, and optionally the second therapeutic agent, to treat, delay the onset, or alleviate OSA in a subject. The kit may further comprise a description of selecting an individual suitable for treatment based on identifying whether that individual has OSA, e.g., following a routine procedure.

The instructions relating to the use of the pharmaceutical composition disclosed herein generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating OSA. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle).

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit, and scope of the present disclosure. All such modifications are intended to be within the scope of the disclosure.

### Example 1: Therapeutic Effects in New Zealand Obese Mouse Model

Obese rodents show features of sleep apnea, including impaired upper airway anatomy or function and changes in breathing patterns during sleep. New Zealand Obese (NZO) mice have increased visceral fat compared to lean mice with fat deposits in the pharyngeal soft tissues, tongue, soft palate, and upper airway, which are similar to observations found in OSA patients. It is reported that NZO mice show sleep disordered breathing. Kim et al., J. Clinical Medicine, 2019, 8:2049. As such, the New Zealand obese mouse model can be used to study OSA.

This example explores the therapeutic effects of the composition provided herein for alleviating symptoms of OSA in NZO mice.

NZO mice are feed with a high-fat (60%) diet to induce obesity for 10 days. Afterwards, electroencephalography (EEG) electrodes are surgically fixed onto the skull of the mice using miniature stainless steel screws. Each EEG electrode is largely wrapped in an insulating material and surrounded by multiple 1-mm long non-insulating tips. The non-insulating tips touch the dura mater and the corresponding EEG electrode is located in the vermis of the cerebellum. An electromyography (EMG) recording electrode is placed in the right acromion muscle of the neck. All electrodes are welded to an acrylic covered connector with only one corrector located outside the mouse body. The connector is linked a signal amplifier to a signal recording device through a wire, which does not restrict the normal activity of the mice. After the mice are fully recovered from the surgery, they are placed in a cage that can shield the electrical signal.

The mice are randomly assigned into the four groups listed in Table 1 below based on their weights and weight changes. Treatment is performed at least one week after the above operation, following the conditions listed in Table 1. The control group mice are treated with saline. The OS1 group mice are treated with a composition comprising curcumin. The OS2 group mice are treated with a composition comprising resveratrol. The OS3 group mice are treated with a composition comprising both curcumin and resveratrol.

TABLE 1
NZO Mice Model Experimental Design

| | Control group | OS1 group | OS2 group | OS3 group |
|---|---|---|---|---|
| | (n = 6) | (n = 6) | (n = 6) | (n = 6) |
| Composition | saline | curcumin | resveratrol | curcumin and |
| | | | | resveratrol |
| Dosage | 4 mL/ kg | 4 mL/ kg | 4 mL/ kg | 4 mL/ kg |
| Applied site | Subcutaneous, | Subcutaneous, | Subcutaneous, | Subcutaneous, |
| | fat deposit area | fat deposit area | fat deposit area | fat deposit area |
| Frequency | at least once | at least once | at least once | at least once |

Polysomnography is performed on the mice based on the EMG result. A whole-body plethysmograph is performed for obtaining respiratory amplitude and rhythm information. The data are to be recorded for 24 consecutive days after injection. The first three days are the adaptation period. Body weights of the mice are measured during the 24-day period, starting from the day when obesity is induced (Day 1). The first three days are the adaptation period. The compositions are given twice on Day 10 and Day 13. At the end of the experiment (Day 24), the mice are to be fasted for 12-14 hours. The mice are then sacrificed and the locations of the injections are removed and weighed.

### Example 2: Therapeutic Effects in Obese Zucker Rat Model

Obese rodents show features of sleep apnea, including impaired upper airway anatomy or function and changes in breathing patterns during sleep. Obese Zucker rats (body weight 600 g) show reduced pharyngeal cross-sectional areas during both expiration and inspiration and a greater fat infiltration in the tongue muscle as compared with non-obese Zucker rats. Kim et al., J. Clinical Medicine, 2019, 8:2049. As such, the New Zealand obese mouse model can be used to study OSA.

This example explores the therapeutic effects of the composition provided herein for alleviating symptoms of OSA in Obese Zucker rats.

Obese Zucker rats are feed with a high-fat (60%) diet to induce obesity for 10 days. Afterwards, electroencephalography (EEG) electrodes are surgically fixed onto the skull of the rats using miniature stainless steel screws. Each EEG electrode is largely wrapped in an insulating material and surrounded by multiple 1-mm long non-insulating tips. The non-insulating tips touch the dura mater and the corresponding EEG electrode is located in the vermis of the cerebellum. An electromyography (EMG) recording electrode is placed in the right acromion muscle of the neck. All electrodes are welded to an acrylic covered connector with only one corrector located outside the mouse body. The connector is linked a signal amplifier to a signal recording device through a wire, which does not restrict the normal activity of the rats. After the rats are fully recovered from the surgery, they are placed in a cage that can shield the electrical signal.

The rats are randomly assigned into the four groups listed in Table 2 below based on their weights and weight changes. Treatment is performed at least one week after the above operation, following the conditions listed in Table 2. The control group rats are treated with saline. The OS1 group mice are treated with a composition comprising curcumin. The OS2 group mice are treated with a composition comprising resveratrol. The OS3 group mice are treated with a composition comprising curcumin and resveratrol.

TABLE 2
Obese Zucker Rat Model Experimental Design

| | Control group | OS1 group | OS2 group | OS3 group |
|---|---|---|---|---|
| | (n = 6) | (n = 6) | (n = 6) | (n = 6) |
| Composition | saline | curcumin | resveratrol | curcumin and |
| | | | | resveratrol |
| Dosage | 4 mL/ kg | 4 mL/ kg | 4 mL/ kg | 4 mL/ kg |
| Applied site | Subcutaneous, | Subcutaneous, | Subcutaneous, | Subcutaneous, |
| | fat deposit area | fat deposit area | fat deposit area | fat deposit area |
| Frequency | at least once | at least once | at least once | at least once |

Polysomnography is performed on the rats based on the EMG result. A whole-body plethysmograph is performed for obtaining respiratory amplitude and rhythm information. The data are to be recorded for 24 consecutive days after injection. The first three days are the adaptation period.

Body weights of the rats are measured during the 24-day period, starting from the day when obesity is induced (Day 1). The first three days are the adaptation period. The compositions are given twice on Day 10 and Day 13. At the end of the experiment (Day 24), the rats are to be fasted for 12-14 hours. The rats are then sacrificed and the locations of the injections are removed and weighed.

### Example 3: Therapeutic Effects in C57BL/6 Mouse Model

The lean C57BL/6 mouse model has been used for studying neuromuscular responses of upper airway and respiratory instability. This example explores the therapeutic effects of the composition provided herein for alleviating symptoms of OSA in C57BL/6 mice.

C57BL/6 mice are fed with a high-fat (60%) diet to induce obesity for 10 days. Afterwards, electroencephalography (EEG) electrodes are surgically fixed onto the skull of the mice using miniature stainless steel screws. Each EEG electrode is largely wrapped in an insulating material and surrounded by multiple 1-mm long non-insulating tips. The non-insulating tips touch the dura mater and the corresponding EEG electrode is located in the vermis of the cerebellum. An electromyography (EMG) recording electrode is placed in the right acromion muscle of the neck. All electrodes are welded to an acrylic covered connector with only one corrector located outside the mouse body. The connector is linked a signal amplifier to a signal recording device through a wire, which does not restrict the normal activity of the mice. After the mice are fully recovered from the surgery, they are placed in a cage that can shield the electrical signal.

The mice are randomly assigned into the four groups listed in Table 3 below based on their weights and weight changes. Treatment is performed at least one week after the above operation, following the conditions listed in Table 3. The control group mice are treated with saline. The OS1 group mice are treated with a composition comprising curcumin. The OS2 group mice are treated with a composition comprising resveratrol. The OS3 group mice are treated with a composition comprising curcumin and resveratrol.

TABLE 3
C57BL/6 Mice Model Experimental Design

| | Control group | OS1 group | OS2 group | OS3 group |
|---|---|---|---|---|
| | (n = 6) | (n = 6) | (n = 6) | (n = 6) |
| Composition | saline | curcumin | resveratrol | curcumin and |
| | | | | resveratrol |
| Dosage | 4 mL/ kg | 4 mL/ kg | 4 mL/ kg | 4 mL/ kg |
| Applied site | Subcutaneous, | Subcutaneous, | Subcutaneous, | Subcutaneous, |
| | fat deposit area | fat deposit area | fat deposit area | fat deposit area |
| Frequency | at least once | at least once | at least once | at least once |

Polysomnography is performed on the mice based on the EMG result. A whole-body plethysmograph is performed for obtaining respiratory amplitude and rhythm information. The data are to be recorded for 24 consecutive days after injection. The first three days are the adaptation period.

Body weights of the mice are measured during the 24-day period, starting from the day when obesity is induced (Day 1). The first three days are the adaptation period. The compositions are given twice on Day 10 and Day 13. At the end of the experiment (Day 24), the mice are to be fasted for 12-14 hours. The mice are then sacrificed and the locations of the injections are removed and weighed.

### Example 4: Evaluation of Lipolysis Efficacy of Exemplary Formulation in Animal Models

Diet-Induced Obesity (DIO) rats are an animal model widely used to study the effects weight loss and other beneficial metabolic properties for therapeutic agent candidates. Gijsen and Kotze-Horstmann., Obesity Research & Clinical Practice, 2023, 17(6):449-457. This example explores the therapeutic effects of an exemplary composition provided herein for reduction of gluteal fat in a DIO SD rat model.

Sprague Dawley (SD) rats were fed 60% high-fat diet (HFD) to induce obesity for 10 days. On the 10^{th} day, the rats are randomized into four groups, each being treated as described in Table 4 below. Prior to injecting the indicated treatment formulation, gluteal fat at the injection site was scanned by micro-CT (pt-CT). In the third week of HFD induction, rats were injected with 4 mL/kg of normal saline for the control group (HFD-C), with 4 mL/kg of OP-A1 (the OP-A1 group), with 4 mL/kg of OP-A2 (the OP-A2 group), and with 4 mL/kg of OP-A (the OP-A group) via subcutaneous injection.

All groups were injected once a day for 4 consecutive days. The study design is summarized in Table 4 below. Details of the tested formulations are provided in Table 5 below.

A second micro-CT ranging from pubis to the coccyx mainly covering the tail fat mass was performed 14 days after the final injection. Micro-CT analysis images were all gray-scale black and white images, and the gray-scale threshold range was 0-255. The test conditions set were the lower gray threshold of adipose tissue to 40 and the upper gray threshold to 85. The image results were analyzed as slice in units of 35 μm. Data analysis results were presented as [adipose volume (defined by gray scale value 40-85)/average fat volume of HFD-C group]*100%.

TABLE 4
Study Design

| | | Diet- | | | | Dose | |
|---|---|---|---|---|---|---|---|
| Study | Animal | Induced | Injection | Test | Dose Level | Volume | Dose |
| Group | no. | Period | Site | Article | (mg/kg) | (mL/kg) | Frequency |
| HFD-C | 13 | 4 weeks | Gluteal | Normal | — | 4 | Once a |
| | | | Fat | Saline | | | day, total 4 |
| OP-A1 | 13 | | | OP-A1 | Equivalent | 4 | doses |
| | | | | | dose to | | |
| | | | | | OP-A | | |
| OP-A2 | 13 | | | OP-A2 | Equivalent | 4 | |
| | | | | | dose to | | |
| | | | | | OP-A | | |
| OP-A | 13 | | | OP-A | 20 | 4 | |

TABLE 5
Formulations

| Group | OP-A | OP-A1 | OP-A2 |
|---|---|---|---|
| Curcumin | 0.4% (4 mg/mL) | 0.4% (4 mg/mL) | — |
| Resveratrol | 0.1% (1 mg/mL) | — | 0.1% (1 mg/mL) |
| ELP | 20% | 16% | 10% |

Fat volume in the treated area was significantly reduced in the OP-A group compared independently to the HFD-C group (placebo/control) (p<0.01) and when compared to the OP-A1 (p<0.05) and OP-A2 groups (p<0.05). There was no statistical difference between OP-A1 or OP-A2 and HFD-C (p>0.05), even though the fat volume in the treated area was decreased in the OP-A1 and OP-A2 groups. The results obtained are provided in Table 6 below.

TABLE 6
Levels of Local Fat Reduction

| Group | HFD-C | OP-A1 | OP-A2 | OP-A |
|---|---|---|---|---|
| Fat Volume in the treated area | 100.0 ± 24.5 | 80.5 ±42.0 | 87.0 ±41.4 | 48.1 ± 34.4 **^{, #, $} |
| (% of HFD-C) | | | | |
| Fat Volume change in the | — | − 19.5 ±42.0 | − 13.0 ±41.4 | −51.9 ± 34.4 **^{, #, $} |
| treated area compared to | | | | |
| HFD-C (%) | | | | |
| Fasting body weight (%) | 100.0 ± 4.0 | 96.8 ± 4.6 | 100.0 ±7.9 | 98.0 ±6.1 |

*p < 0.05,
** p < 0.01,
***p < 0.001 compared with HFD-C.
^{#} p < 0.05,
^{##}p < 0.01,
^{###}p < 0.001 compared with OP-A1.
^{$} p < 0.05,
^{$$}p < 0.01,
^{$$$}p < 0.001 compared with OP-A2.

### Example 5: A Single-Dose Toxicity Study of Exemplary Pharmaceutical Compositions Following Tongue Injection Administration with a 3-Week Recovery Period in Healthy Mini-Pigs

This example evaluates the safety of an exemplary pharmaceutical composition injected through the tongue in healthy, male mini-pigs by a single dose. In this study, each mini-pig was administered a single dose of the exemplary pharmaceutical composition at 10 mg/animal (low dose group) or 20 mg/animal (high dose group), or administered a vehicle (control group). The doses provided herein refer to the amount of the active agent in the pharmaceutical composition tested herein (curcumin and resveratrol in this case). The animals were investigated in a 3-week recovery period following the administration for multiple safety parameters assessment.

The exemplary pharmaceutical composition investigated in this study comprises 0.4% (4 mg/mL) curcumin, 0.1% (1 mg/mL) resveratrol, and 16% ELP, corresponding to OP-A. See Table 5 above.

### I. Clinical Observations

No pharmaceutical composition-related abnormal clinical signs of toxicity were noted at both doses.

### II. Body Weight

Tables 8A and 8B below summarize the observed body weight changes over the study period.

TABLE 8A
Summary of Body Weight (kg)

| | | Day(s) Relative to Start Date | | | | | | |
|---|---|---|---|---|---|---|---|---|
| Sex: Male | | 1(am) | 4(am) | 8(am) | 11(am) | 15(am) | 18(am) | 21(pm) |
| 0 mg/ | Mean | 11.967 | 12.147 | 12.580 | 12.917 | 13.713 | 13.987 | 15.270 |
| animal | SD | 0.514 | 0.669 | 0.721 | 0.689 | 0.745 | 0.761 | 0.951 |
| | N | 3 | 3 | 3 | 3 | 3 | 3 | 3 |
| 10 mg/ | Mean | 11.927 | 12.143 | 12.860 | 13.340 | 13.877 | 14.243 | 15.367 |
| animal | SD | 0.365 | 0.160 | 0.246 | 0.193 | 0.223 | 0.166 | 0.216 |
| | N | 3 | 3 | 3 | 3 | 3 | 3 | 3 |
| 20 mg/ | Mean | 11.873 | 11.910 | 12.457 | 13.063 | 13.670 | 13.840 | 14.740 |
| animal | SD | 0.283 | 0.407 | 0.732 | 0.600 | 0.435 | 0.469 | 0.401 |
| | N | 3 | 3 | 3 | 3 | 3 | 3 | 3 |

TABLE 8B
Summary of Body Weight Changes

| | | Day(s) Relative to Start Date | | | | | | | | |
|---|---|---|---|---|---|---|---|---|---|---|
| | | | | | | | | | Abs | |
| | | Base | Abs | Abs | Abs | Abs | Abs | Abs | Gain | % |
| | | Wt. | Gain | Gain | Gain | Gain | Gain | Gain | Total | Gain |
| | | [a] | [a] | [a] | [a] | [a] | [a] | [a] | [a] | [a] |
| Sex: Male | | 1 | 1→4 | 4→8 | 8→11 | 11→15 | 15→18 | 18→21 | 1→21 | 1→21 |
| 0 mg/ | Mean | 11.967 | 0.180 | 0.433 | 0.337 | 0.797 | 0.273 | 1.283 | 3.303 | 27.54 |
| animal | SD | 0.514 | 0.246 | 0.072 | 0.08 | 0.160 | 0.051 | 0.290 | 0.453 | 2.79 |
| | N | 3 | 3 | 3 | 3 | 3 | 3 | 3 | 3 | 3 |
| 10 mg/ | Mean | 11.927 | 0.217 | 0.717 | 0.480 | 0.537 | 0.367 | 1.123 | 3.440 | 28.89 |
| animal | SD | 0.365 | 0.353 | 0.218 | 0.087 | 0.076 | 0.071 | 0.064 | 0.165 | 2.20 |
| | N | 3 | 3 | 3 | 3 | 3 | 3 | 3 | 3 | 3 |
| 20 mg/ | Mean | 11.873 | 0.037 | 0.547 | 0.607 | 0.607 | 0.170 | 0.900 | 2.867 | 24.14 |
| animal | SD | 0.283 | 0.170 | 0.325 | 0.176 | 0.168 | 0.060 | 0.095 | 0.210 | 1.67 |
| | N | 3 | 3 | 3 | 3 | 3 | 3 | 3 | 3 | 3 |

Overall, no pharmaceutical composition-related alteration in body weight was noted in both the low dose and high dose groups, as relative to the control group.

### III. Food Consumption

No pharmaceutical composition-related alteration in food consumption was noted in both the low dose and high dose groups as compared with the control group.

### IV. Pulse Oximetry

No pharmaceutical composition-related change in pulse oximetry parameters was noted in both the low dose and high dose groups as compared with the control group. See Table 9 below.

TABLE 9
Summary of Pulse Oximetry

| | | Day(s) Relative to Start Date | | | | | |
|---|---|---|---|---|---|---|---|
| Sex: Male | | 1(am) | 1(4.0 hr) | 1(8.0 hr) | 2(24 hr) | 3(48 hr) | 4(72 hr) |
| 0 mg / | Mean | 99.1 | 96.8 | 98.0 | 96.9 | 99.7 | 97.8 |
| animal | SD | 0.2 | 1.8 | 0.9 | 0.4 | 0.3 | 1.3 |
| | N | 3 | 3 | 3 | 3 | 3 | 3 |
| 10 mg/ | Mean | 99.2 | 96.6 | 97.4 | 98.0 | 99.2 | 98.8 |
| animal | SD | 0.8 | 1.1 | 1.8 | 1.5 | 0.7 | 1.6 |
| | N | 3 | 3 | 3 | 3 | 3 | 3 |
| 20 mg/ | Mean | 98.0 | 96.6 | 98.8 | 97.8 | 97.3 | 98.6 |
| animal | SD | 1.2 | 1.1 | 0.5 | 1.6 | 1.8 | 0.8 |
| | N | 3 | 3 | 3 | 3 | 3 | 3 |

### V. Respiratory Evaluation

Both low dose and high dose groups exhibited no change in the respiratory parameters during the first 72 hours after administration, including tidal volume (measured as the amount of air inhaled or exhaled during a normal breath), minute volume (measured as the volume of air breathed in one minute), and respiratory rate (breaths per minute) were noted, as relative to the control group. See Tables 10A-10C below.

TABLE 10A
Summary of Tidal Volume in Milliliters

| | | Day(s) Relative to Start Date | | | | | |
|---|---|---|---|---|---|---|---|
| Sex: Male | | 1(PreD) | 1(4.0 hr) | 1(8.0 hr) | 2(24 hr) | 3(48 hr) | 4(72 hr) |
| 0 mg/ | Mean | 91.7 | 139.3 | 108.0 | 129.7 | 103.0 | 76.0 |
| animal | SD | 45.7 | 7.0 | 10.8 | 21.4 | 19.0 | 16.1 |
| | N | 3 | 3 | 3 | 3 | 3 | 3 |
| 10 mg/ | Mean | 85.0 | 136.7 | 100.7 | 111.0 | 99.0 | 96.3 |
| animal | SD | 4.0 | 34.8 | 15.3 | 12.8 | 17.3 | 29.5 |
| | N | 3 | 3 | 3 | 3 | 3 | 3 |
| 20 mg/ | Mean | 88.0 | 142.3 | 106.7 | 146.7 | 120.3 | 119.7 |
| animal | SD | 30.5 | 21.5 | 37.3 | 31.0 | 53.5 | 53.7 |
| | N | 3 | 3 | 3 | 3 | 3 | 3 |

*PreD: Pre-dosing

TABLE 10B
Summary of Minute Volume in Milliliters

| | | Day(s) Relative to Start Date | | | | | |
|---|---|---|---|---|---|---|---|
| Sex: Male | | 1(PreD) | 1(4.0 hr) | 1(8.0 hr) | 2(24 hr) | 3(48 hr) | 4(72 hr) |
| 0 mg/ | Mean | 4519.0 | 2941.3 | 3308.3 | 3297.3 | 2979.0 | 2722.7 |
| animal | SD | 1351.9 | 1156.5 | 295.2 | 457.4 | 391.5 | 1256.7 |
| | N | 3 | 3 | 3 | 3 | 3 | 3 |
| 10 mg/ | Mean | 3147.3 | 2780.7 | 2713.0 | 2798.0 | 3189.0 | 2561.7 |
| animal | SD | 571.9 | 1040.1 | 912.1 | 805.0 | 443.9 | 913.5 |
| | N | 3 | 3 | 3 | 3 | 3 | 3 |
| 20 mg/ | Mean | 4298.7 | 3059.7 | 4045.0 | 3914.7 | 3925.7* | 3504.7 |
| animal | SD | 1217.7 | 382.7 | 1261.6 | 975.5 | 216.5 | 742.7 |
| | N | 3 | 3 | 3 | 3 | 3 | 3 |

*PreD: Pre-dosing

TABLE 10C
Summary of Respiratory Rate⁻¹ (Breaths per minute)

| | | Day(s) Relative to Start Date | | | | | |
|---|---|---|---|---|---|---|---|
| Sex: Male | | 1(PreD) | 1(4.0 hr) | 1(8.0 hr) | 2(24 hr) | 3(48 hr) | 4(72 hr) |
| 0 mg/ | Mean | 52.3 | 21.3 | 31.0 | 25.7 | 30.0 | 36.3 |
| animal | SD | 10.0 | 7.6 | 4.6 | 2.1 | 8.7 | 18.0 |
| | N | 3 | 3 | 3 | 3 | 3 | 3 |
| 10 mg/ | Mean | 37.0 | 20.0 | 26.7 | 25.0 | 32.3 | 27.0 |
| animal | SD | 6.1 | 2.6 | 6.7 | 4.4 | 1.5 | 8.5 |
| | N | 3 | 3 | 3 | 3 | 3 | 3 |
| 20 mg/ | Mean | 56.0 | 22.0 | 46.0 | 27.0 | 39.0 | 34.0 |
| animal | SD | 32.1 | 6.0 | 34.6 | 7.0 | 22.1 | 16.6 |
| | N | 3 | 3 | 3 | 3 | 3 | 3 |

*PreD: Pre-dosing

Although the pigs administered 20 mg of the composition showed a higher minute volume compared to controls 48 h after administering the composition, this was not considered related to the treatment since it was comparable to its corresponding pre-dose levels (Table mB).

### VI. Clinical Chemistry

Except for a decrease in triglyceride (TG) levels in animals of the high dose group, no other pharmaceutical composition-related change in clinical chemistry was observed, as shown in Tables 11 below.

TABLE 11
Summary of Clinical Chemistry Parameters

| Sex: Male | | | 0 mg/ | 10 mg/ | 20 mg/ |
|---|---|---|---|---|---|
| Day(s) Relative to Start Date | | | animal | animal | animal |
| ALT(U/L) | −2[a] | Mean | 50.7 | 41.7 | 46.7 |
| | | SD | 24.0 | 4.7 | 5.1 |
| | | N | 3 | 3 | 3 |
| | 22[a] | Mean | 54.3 | 55.7 | 46.0 |
| | | SD | 31.2 | 10.7 | 12.0 |
| | | N | 3 | 3 | 3 |
| AST(U/L) | −2[a] | Mean | 28.3 | 21.0 | 30.0 |
| | | SD | 2.9 | 3.6 | 4.4 |
| | | N | 3 | 3 | 3 |
| | 22[a] | Mean | 104.0 | 35.0 | 38.7 |
| | | SD | 73.7 | 3.6 | 12.4 |
| | | N | 3 | 3 | 3 |
| ALP(U/L) | −2[a] | Mean | 157.3 | 211.0 | 177.0 |
| | | SD | 57.0 | 78.0 | 36.0 |
| | | N | 3 | 3 | 3 |
| | 22[a] | Mean | 203.3 | 260.3 | 187.7 |
| | | SD | 46.2 | 30.0 | 29.5 |
| | | N | 3 | 3 | 3 |
| GGT(U/L) | −2[a] | Mean | 53.80 | 50.23 | 62.47 |
| | | SD | 10.32 | 0.60 | 18.94 |
| | | N | 3 | 3 | 3 |
| | 22[a] | Mean | 64.70 | 61.93 | 64.03 |
| | | SD | 1.84 | 3.00 | 19.21 |
| | | N | 3 | 3 | 3 |
| CK(U/L) | −2[a] | Mean | 272.0 | 224.0 | 283.0 |
| | | SD | 134.1 | 35.7 | 69.3 |
| | | N | 3 | 3 | 3 |
| | 22[a] | Mean | 4869.7 | 487.0 | 455.0 |
| | | SD | 4559.4 | 122.1 | 258.5 |
| | | N | 3 | 3 | 3 |
| LDH(U/L) | −2[a] | Mean | 470.27 | 420.47 | 481.50 |
| | | SD | 85.23 | 29.08 | 50.75 |
| | | N | 3 | 3 | 3 |
| | 22[a] | Mean | 809.67 | 468.13 | 492.70 |
| | | SD | 417.38 | 23.55 | 131.05 |
| | | N | 3 | 3 | 3 |
| TP(g/L) | −2[a] | Mean | 63.83 | 56.60 | 59.47 |
| | | SD | 1.16 | 1.65 | 5.63 |
| | | N | 3 | 3 | 3 |
| | 22[a] | Mean | 71.50 | 61.23* | 70.87 |
| | | SD | 3.58 | 1.71 | 5.02 |
| | | N | 3 | 3 | 3 |
| ALB(g/L) | −2[a] | Mean | 33.70 | 33.80 | 34.90 |
| | | SD | 2.01 | 1.83 | 4.45 |
| | | N | 3 | 3 | 3 |
| | 22[a] | Mean | 42.40 | 38.00 | 42.07 |
| | | SD | 1.08 | 2.16 | 5.58 |
| | | N | 3 | 3 | 3 |
| GLO(g/L) | −2[a] | Mean | 30.13 | 22.80 | 24.57 |
| | | SD | 2.80 | 0.56 | 4.79 |
| | | N | 3 | 3 | 3 |
| | 22[a] | Mean | 29.10 | 23.23 | 28.80 |
| | | SD | 4.65 | 0.59 | 2.71 |
| | | N | 3 | 3 | 3 |
| A/G(Ratio) | −2[a] | Mean | 1.127 | 1.483 | 1.467 |
| | | SD | 0.166 | 0.101 | 0.377 |
| | | N | 3 | 3 | 3 |
| | 22[a] | Mean | 1.487 | 1.637 | 1.477 |
| | | SD | 0.266 | 0.130 | 0.275 |
| | | N | 3 | 3 | 3 |
| TBILI(μmol/L) | −2[a] | Mean | 2.67 | 2.6 | 2.60 |
| | | SD | 0.15 | 0.17 | 0.17 |
| | | N | 3 | 3 | 3 |
| | 22[a] | Mean | 2.20 | 2.03 | 2.23 |
| | | SD | 0.00 | 0.06 | 0.21 |
| | | N | 3 | 3 | 3 |
| BU(mmol/L) | −2[a] | Mean | 2.657 | 2.350 | 2.923 |
| | | SD | 0.366 | 0.324 | 0.106 |
| | | N | 3 | 3 | 3 |
| | 22[a] | Mean | 2.233 | 2.463 | 2.200 |
| | | SD | 0.384 | 0.535 | 0.483 |
| | | N | 3 | 3 | 3 |
| CRE(μmol/L) | −2[a] | Mean | 45.3 | 44.7 | 39.7 |
| | | SD | 10.1 | 4.5 | 9.5 |
| | | N | 3 | 3 | 3 |
| | 22[a] | Mean | 51.7 | 56.3 | 46.3 |
| | | SD | 15.5 | 13.3 | 9.9 |
| | | N | 3 | 3 | 3 |
| BUN/C(Ratio) | −2[a] | Mean | 15.223 | 13.137 | 19.097 |
| | | SD | 4.704 | 2.266 | 5.235 |
| | | N | 3 | 3 | 3 |
| | 22[a] | Mean | 11.037 | 10.903 | 12.573 |
| | | SD | 1.678 | 0.651 | 5.788 |
| | | N | 3 | 3 | 3 |
| GLU(mmol/L) | −2[a] | Mean | 3.883 | 3.700 | 3.733 |
| | | SD | 0.933 | 0.265 | 0.860 |
| | | N | 3 | 3 | 3 |
| | 22[a] | Mean | 5.150 | 6.027 | 4.493 |
| | | SD | 0.554 | 0.465 | 0.855 |
| | | N | 3 | 3 | 3 |
| CHO(mmol/L) | −2[a] | Mean | 2.180 | 2.150 | 1.913 |
| | | SD | 0.262 | 0.104 | 0.015 |
| | | N | 3 | 3 | 3 |
| | 22[a] | Mean | 2.123 | 2.817 | 2.400 |
| | | SD | 0.439 | 0.391 | 0.079 |
| | | N | 3 | 3 | 3 |
| TG(mmol/L) | −2[a] | Mean | 0.313 | 0.227 | 0.223 |
| | | SD | 0.012 | 0.096 | 0.035 |
| | | N | 3 | 3 | 3 |
| | 22[a] | Mean | 0.217 | 0.213 | 0.053 |
| | | SD | 0.091 | 0.083 | 0.015 |
| | | N | 3 | 3 | 3 |
| Na(mmol/L) | −2[a] | Mean | 141.30 | 139.83 | 142.00 |
| | | SD | 1.97 | 1.96 | 0.79 |
| | | N | 3 | 3 | 3 |
| | 22[a] | Mean | 142.70 | 143.13 | 144.63 |
| | | SD | 1.25 | 2.00 | 2.83 |
| | | N | 3 | 3 | 3 |
| K(mmol/L) | −2[a] | Mean | 5.093 | 4.833 | 4.903 |
| | | SD | 0.351 | 0.620 | 0.112 |
| | | N | 3 | 3 | 3 |
| | 22[a] | Mean | 5.047 | 5.013 | 4.807 |
| | | SD | 1.035 | 0.569 | 0.845 |
| | | N | 3 | 3 | 3 |
| Cl(mmol/L) | −2[a] | Mean | 100.0 | 103.07 | 102.53 |
| | | SD | 2.27 | 0.78 | 1.46 |
| | | N | 3 | 3 | 3 |
| | 22[a] | Mean | 99.13 | 98.30 | 97.90 |
| | | SD | 0.35 | 1.37 | 0.85 |
| | | N | 3 | 3 | 3 |
| Ca(mmol/L) | −2[a] | Mean | 2.473 | 2.540 | 2.483 |
| | | SD | 0.086 | 0.026 | 0.108 |
| | | N | 3 | 3 | 3 |
| | 22[a] | Mean | 2.563 | 2.510 | 2.620 |
| | | SD | 0.038 | 0.131 | 0.072 |
| | | N | 3 | 3 | 3 |
| P(mmol/L) | −2[a] | Mean | 2.5 | 2.57 | 2.50 |
| | | SD | 0.36 | 0.42 | 0.00 |
| | | N | 3 | 3 | 3 |
| | 22[a] | Mean | 2.70 | 2.73 | 2.73 |
| | | SD | 0.35 | 0.23 | 0.06 |
| | | N | 3 | 3 | 3 |

### VII. Coagulation

No pharmaceutical composition-related change in coagulation parameters was noted.

### VIII. Organ Weights

No pharmaceutical composition-related change in organ weight, or organ-to-body weight and organ-to-brain weight ratios was noted.

### IX. Gross Pathology

No pharmaceutical composition-related changes in gross pathology, as determined by macroscopic observations, were noted. Macroscopic observations were considered incidental findings and not related to pharmaceutical composition.

### X. Histopathology

No pharmaceutical composition-related change in histopathology was observed as determined by microscopic observations.

In sum, no sign of toxicity was observed in the mini-pigs treated with the pharmaceutical composition at either dose throughout the study. Further, no abnormal pattern was noted in food consumption, pulse oximetry parameters, respiratory parameters, clinical chemistry parameters, hematology parameters, coagulation parameters, organ weight parameters, macroscopic and microscopic observations. A decrease in triglyceride levels was observed in animals administered the 20 mg/animal dose (high dose).

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method for alleviating a symptom associated with obstructive sleep apnea (GSA), the method comprising administering to a subject in need thereof an effective amount of a pharmaceutical composition, which comprises:
(a) a resveratrol compound and/or a curcumin compound; and
(b) a pharmaceutically acceptable carrier.

2. The method of claim 1, wherein the subject is a human patient suffering from OSA.

3. The method of claim 1, wherein the symptom associated with OSA comprises loud and disruptive snoring, witnessed apneas during sleep, excessive daytime sleeping, morning headaches, waking during the night and gasping or choking, awakening in the morning with a dry mouth and/or sore throat.

4. The method of claim 1, wherein the pharmaceutical composition comprises resveratrol and/or curcumin in (a).

5. The method of claim 4, wherein the pharmaceutical composition comprises both resveratrol and curcumin.

6. The method of claim 1, wherein the pharmaceutically acceptable carrier comprises a first pharmaceutically acceptable non-ionic surfactant having a hydrophilic-lipophilic balance value (HLB value) greater than 9.

7. The method of claim 6, wherein the first pharmaceutically acceptable non-ionic surfactant comprises Polyoxyl 40 Stearate, Polyoxyl 20 Cetostearyl Ether, Polyoxyl 12 Cetostearyl Ether, Tween 80, polyoxyl 15 hydroxystearate (solutol HS 15), a polyoxyethylene derivative, a polyoxyethylene castor oil derivative, or a combination thereof.

8. The method of claim 6, wherein the first pharmaceutically acceptable non-ionic surfactant forms a first plurality of micelles with the resveratrol compound and/or the curcumin compound.

9. The method of claim 6, wherein the weight ratio of the resveratrol compound and/or the curcumin compound to the first pharmaceutically acceptable non-ionic surfactant in the composition is 1:2-1:500.

10. The method of claim 8, wherein the micelles in the first plurality of micelles have a diameter ranging from about 1 nm to about 250 nm; and/or a polydispersity index (PDI) value less than 0.4.

11. The method of claim 10, wherein the diameter ranges from about 5 nm to about 50 nm.

12. The method of claim 9, wherein the first plurality of micelles encompasses both the resveratrol compound and the curcumin compound.

13. The method of claim 1, wherein the pharmaceutically acceptable carrier further comprises a second pharmaceutically acceptable non-ionic surfactant having a hydrophilic-lipophilic balance value (HLB value) greater than 9.

14. The method of claim 13, wherein the first pharmaceutically acceptable non-ionic surfactant comprises Polyoxyl 40 Stearate, Polyoxyl 20 Cetostearyl Ether, Polyoxyl 12 Cetostearyl Ether, Tween 80, polyoxyl 15 hydroxystearate (solutol HS 15), a polyoxyethylene derivative, a polyoxyethylene castor oil derivative, or a combination thereof.

15. The method of claim 13, wherein the first plurality of micelles is formed by the first pharmaceutically acceptable non-ionic surfactant and the resveratrol compound; and wherein the second pharmaceutically acceptable non-ionic surfactant and the curcumin compound form a second plurality of micelles.

16. The method of claim 15, wherein the weight ratio of the curcumin compound to the second pharmaceutically acceptable non-ionic surfactant in the composition is 1:2-1:500.

17. The method of claim 15, wherein the micelles in the second plurality of micelles have a diameter ranging from about 1 nm to about 250 nm, and/or a polydispersity index (PDI) value less than 0.4.

18. The method of claim 17, wherein the diameter ranges from about 5 nm to about 50 nm.

19. The method of claim 1, wherein the pharmaceutical composition further comprises a cosolvent, a suspending agent, an oil phase excipient, or a combination thereof.

20. The method of claim 1, wherein the pharmaceutical composition is administered to the subject via injection.

21. The method of claim 20, wherein the injection is subcutaneous injection.

22. The method of claim 1, wherein the pharmaceutical composition is administered to the tongue or a perioral area of the subject.

23. The method of claim 21, wherein the pharmaceutical composition is administered by sublingual administration.
